(19) United States
(12) Patent Application Publication
(10) Pub. No.: US 2023/0190678 A1
(43) Pub. Date: Jun. 22, 2023

(54) DELAYED RELEASE COMPOSITION COMPRISING ENTERIC COATED DRUG LOADED IN PSYLLIUM HUSK MATRIX

(71) Applicants: Vikram Andrew NAHARWAR, Mumbai (IN); Tresha NAHARWAR, Mumbai (IN)

(72) Inventors: Vikram Andrew NAHARWAR, Mumbai (IN); Tresha NAHARWAR, Mumbai (IN)

(21) Appl. No.: 17/915,216

(22) PCT Filed: Mar. 2, 2022
(86) PCT No.: PCT/IB2022/051831

§ 371 Date: Sep. 28, 2022

(30) Foreign Application Priority Data
Jan. 31, 2021 (IN) 202121004193

(51) Int. Cl. A61K 31/12; A61K 9/50; A61K 47/46; A61K 47/10

(52) U.S. Cl. CPC A61K 31/12; A61K 9/5047; A61K 47/10; A61K 47/46

(57) ABSTRACT

The present invention relates to a delayed release composition comprising enteric coated drug loaded in psyllium husk matrix. The invention specifically provides curcumin loaded in psyllium husk matrix with 25% ethanol solvent used for the preparation of composition which is coated with enteric polymer like hydroxypropyl methyl cellulose.

### PRIORITY

This application claims the benefit of Indian Complete Application Number 202121004193 dated 31^{st} January November 2021 entitled, ‘Delayed release composition comprising enteric coated drug loaded in psyllium husk matrix’, the contents of which are incorporated herein by reference.

### FIELD OF THE INVENTION

The present invention relates to a delayed release composition comprising enteric coated drug loaded in psyllium husk matrix. The invention specifically provides curcumin loaded in psyllium husk matrix with 25% ethanol solvent used for the preparation of composition which is coated with enteric polymer like hydroxypropyl methyl cellulose.

### BACKGROUND OF THE INVENTION

Ispaghula (Plantago ovata Forskal) seeds contain mucilage in the epidermis of the seeds. The polysaccharides in the mucilage of these seeds constitute a diverse class of biological macromolecules with a broad range of physiochemical properties which are widely used for various applications in Pharmacy and medicine.

A traditional plant known as ‘ISABGOL’ is widely used as home remedy in all cultures, in various kinds of diseases, Conditions like chronic constipation, diarrhea, inflammation of mucous membrane of GI and genitourinary tracts, duodenal ulcer, gonorrhea, piles, etc., as bulk forming, non-irritant laxative drug, demulcent, as a cervical dilator etc.

A large number of carbohydrates containing excipients are available from natural Sources have their own place due to variety of properties offers by them, as they are widely used as binding agents, coating materials, suspending agents, granulating agents, easily dispersible material, increasing viscosity of aqueous solution in pharmaceutical industry. Natural carbohydrates, polymers are hydrocolloids, used as gel forming components, sweetener, binder, flavoring agents, lubricants, taste masking agents to prepare easy to swallow compositions. One of the trends in this area is of study the useful substances of natural origin, for such substances tend to be biodegradable, biocompatible and non-toxic. The literature survey reveals that ‘Mucilage’ obtained from plantago ovata husk has greatly attracted the attention of Researcher workers, in this area during last decades. Till date natural carbohydrate are used in modern dosage form for release control, also in the form of matrix material, encapsulating excipients, coating material, on as a carrier to the target the drug to tissue or site-specific drug delivery system.

It was observed that a daily dose of 10.5 g of ispaghula was well tolerated and the majority of adverse events recorded were minor, of short duration and either unrelated or possibly related to the study treatment. The results from the study suggested that ispaghula husk could be used with confidence for the long-term treatment of mild-to-moderate hypercholesterolemia. US Food and Drug Administration recently authorized the use of health claims on food products containing soluble fiber from psyllium that state that they are associated with a decreased risk of coronary heart disease. The addition of psyllium to a traditional diet for Persons with diabetes is safe, is well tolerated, and improves glycemic and Lipid control in men with type 2 diabetes and hypercholesterolemia. The Efficacy and safety of traditional medical therapies for chronic constipation. Reactions may also occur from breathing in the dust or from skin contact it is said that it is boon for patients and bane for providers.

WO2015/087259 discloses colon specific delayed release pharmaceutical compositions comprising: a) a matrix consisting of hydrophilic substances wherein curcumin is dispersed; b) a gastro resistant or acid resistant pH independent coating with a lag time of matrix a). The compositions according to the invention may also contain other active ingredients with synergic, complementary or otherwise useful activities. Examples of said active ingredients include probiotics (lactobacilli, bifidobacteria), digestive enzymes (enteric juices), prebiotics (butyrates, propionates, medium-long chain fatty acids, omega-3 fatty acids or esters), fibres (psyllium, guar gum, acacia fibres, calcium polycarbophil), antispastics (trimebutine and the salts thereof.

Curcumin helps to decrease ulcers or any infection in the digestive tract when combined with the digestive properties of Psyllium Husk. One such product is available by myeast-wellness as combination.

However, formulating the combination and delivering the drug at release site difficult because of the higher amount of psyllium husk needed.

The current inventors have developed a delayed release composition comprising enteric coated drug loaded in psyllium husk matrix.

The composition developed according to the invention is beneficial to deliver the drug in colon for local treatment as well as systemic treatment.

### OBJECT OF THE INVENTION

The main object of the invention is to provide a delayed release composition comprising enteric coated drug loaded in psyllium husk matrix.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

The subject matter disclosed and claimed herein, in one embodiment thereof, comprises a delayed release composition comprising enteric coated drug loaded in psyllium husk matrix.

In an aspect of the embodiment, the model drug is curcumin.

In another aspect of the embodiment, the ratio of psyllium husk to drug is 1:1

In yet another aspect of the embodiment, the composition comprises a solvent to prepare the matrix. In a specific aspect of the embodiment, the solvent is ethanol or methanol or acetone. In a further aspect of the embodiment, the solvent is 25% ethanol.

In one of the aspects of the embodiment, the composition provides release of drug in colon and ileum at a pH between 6.8 to 7.2

In yet another aspect of the embodiment, the drug loaded in the psyllium husk matrix is more than 65%.

In a specific aspect of the embodiment, the composition is in the form of granules.

In another embodiment, the present invention relates to method for preparing a delayed release composition comprising enteric coated drug loaded in psyllium husk matrix, the method comprising
a. Mixing of drug and psyllium husk to form a mixture;
b. Adding solvent to the mixture under pressure to form a matrix;
c. Reducing the temperature of the matrix to allow maximum swelling of mucilage and absorption of the drug into the matrix.

In an aspect of the embodiment, the temperature of the matrix is reduced to 10 degree.

### DETAILED DESCRIPTION OF THE INVENTION

The present invention relates to a delayed release composition comprising enteric coated drug loaded in psyllium husk matrix. The invention specifically provides curcumin loaded in psyllium husk matrix with 25% ethanol solvent used for the preparation of composition which is coated with enteric polymer like hydroxypropyl methyl cellulose.

The present invention will now be described with reference to the accompanying embodiments of the invention. This invention may, however, be embodied in many different forms and should not be construed as being limited to the embodiment set forth herein. Rather, the embodiment is provided so that this disclosure will be thorough, and will fully convey the scope of the invention to those skilled in the art.

The terms used in the specification are defined as follows.

As used herein, the term “about” means that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, unrecited elements or method steps.

A “subject,” “individual,” or “patient,” is used interchangeably herein, which refers to a vertebrate, preferably a mammal, more preferably a human. Tissues, cells and their progeny of a biological entity obtained in vitro or cultured in vitro are also encompassed.

The use of the terms “a” and “an” and “the” and similar referents in the context of describing the elements (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential to the practice of the invention.

As used herein, the terms “treat,” “treated,” or “treating” mean both therapeutic treatment or prophylactic or preventative measures wherein the object is to prevent or slow down (lessen) an undesired physiological condition, disorder or disease, or obtain beneficial or desired clinical results. For purposes of this invention, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease.

The subject matter disclosed and claimed herein, in one embodiment thereof, comprises a delayed release composition comprising enteric coated drug loaded in psyllium husk matrix.

In a specific aspect of the embodiment, the composition is in the form of granules.

In an aspect of the embodiment, the model drug is curcumin.

In another aspect of the embodiment, the ratio of psyllium husk to drug is 1:1

In yet another aspect of the embodiment, the composition comprises a solvent to prepare the matrix. In a specific aspect of the embodiment, the solvent is ethanol or methanol or acetone. The different solvents can be used in different concentrations like 100, 75, 50 and 25%.

In a further aspect of the embodiment, the solvent is 25% ethanol.

A colonic delivery refers to delivery of drugs accurately into the lower GI tract (by avoiding the drug release in upper GIT), which occurs primarily in the large intestine (i.e. colon). Rectal administration is another route used for colon targeting, but it shows less compliance (uncomfortable) and becomes difficult to reach the colon. Conventional dosage forms that are used in the prevention of colon diseases (ulcerative colitis, crohn’s diseases, amoebiasis) are failing as an improper amount of drug reaches site of action. Conventional dosage form affords the drug to be absorbed from the upper part of GIT, i.e., stomach. This action of conventional dosage form has a serious drawback for colonic localized delivery. Thus, for efficient and safe therapy, the drug is needed to be preserve from upper hostile environment.

Site-specific delivery into the colon is not only needed for local treatment of a variety of colon diseases, like ulcerative colitis, Chron’s diseases, amoebiasis, colon cancer, but also systemic delivery of proteins and peptides this is because of less diversity and intensity of digestive enzymes and less proteolytic activity of colon mucosa than that observed in the small intestine. Beside the colon diseases, this system is also helpful in the treatment of asthma, angina and rheumatoid arthritis for taking advantage of chronotherapeutic drug delivery and for delivery of steroids.

The present invention provides an easier way to deliver these drugs using the composition of the invention and provides both localized and systemic action of the drugs.

An enteric coating is a polymer barrier applied on oral medication. This helps by protecting drugs from the pH (i.e. acidity) of the stomach.

An enteric coating is a barrier that controls the location of oral medication in the digestive system where it is absorbed. The word “enteric” indicates small intestine; therefore, enteric coatings prevent the release of medication before it reaches the small intestine.

Enteric coatings concepts depend on the pH value. Normally pH of stomach is highly acidic, which is nearly 1.2 to 1.4 and pH of intestine is basic. Enteric coating material remains unchanged at the acidic medium that means in the stomach but start to dissolve at the basic medium that means in the intestine. The enteric coated polymers remain unionization at low pH and therefore remain insoluble. But as the pH increases in the GIT, the acidic functional groups are capable of ionization, and the polymer swells or becomes soluble in the intestinal fluid.

In this composition, curcumin is intended to use for the local treatment at the site of intestine and secondly curcumin is not stable in acidic medium thus it was necessary to protect with enteric coating.

In one of the aspects of the embodiment, the composition provides release of drug in colon and ileum at a pH between 6.8 to 7.2

In yet another aspect of the embodiment, the drug loaded in the psyllium husk matrix is more than 65%.

The drug loading in the psyllium husk is dependent on various parameters and processing of the composition as well as the solvent used for the swelling of the matrix. The inventors of the resent invention have optimized the composition as well as the process to provide the most effective composition.

In another embodiment, the present invention relates to method for preparing a delayed release composition comprising enteric coated drug loaded in psyllium husk matrix, the method comprising
a. Mixing of drug and psyllium husk to form a mixture;
b. Adding solvent to the mixture under pressure to form a matrix;
c. Reducing the temperature of the matrix to allow maximum swelling of mucilage and absorption of the drug into the matrix.

In an aspect of the embodiment, the temperature of the matrix is reduced to 10 degree.

It will be understood that various modifications may be made to the aspects disclosed herein. Therefore, the above description should not be construed as limiting, but merely as exemplifications of preferred embodiments. Other arrangements and methods may be implemented by those skilled in the art without departing from the scope and spirit of this invention.

### EXAMPLES

Three different solvents and four methods were experimented to develop best oral formulation which will be effective in intestinal region.

Psyllium husk and curcumin were obtained from commercial sources taken in a weight ratio of 1:1 to develop the composition according to the invention. 40 ml of solvent were used to prepare the matrix.

Method 1 - Curcumin, psyllium husk and appropriate solvent were mixed to form a mixture. The solvent was allowed to evaporate completely to provide a matrix.

Method 2 - Curcumin and psyllium husk were mixed well and the solvent was added to this mixture. The solvents were added while stirring and the solvent was allowed to evaporate completely.

Method 3 - Psyllium husk was mixed with solvent to allow swelling of the husk by soaking within the solvent. After the complete soaking of the solvent, curcumin was added and stirred well.

Method 4 - Psyllium husk and curcumin were mixed gently and thoroughly. The solvent was incorporated under predetermined pressure. The temperature of the matrix was gradually reduced to 10° for maximum swelling of husk mucilage and absorption of curcumin in the matrix.

The dried mixture was sieved and coated with an enteric coating. The biodegradable polymers like hydroxypropyl methylcellulose, hydroxypropyl cellulose, methylcellulose were employed for trial. Each of polymer was dissolved in volatile organic solvent to prepare coating solution. The coating solution was sprayed over the granules in a small coating pan with continuous hot air flow. The coating pan was allowed to rotate until the solvent evaporated and granules dried.

The developed formulations were evaluated for their loading percentage of curcumin in the swollen husk. The loading percentage for each method with each solvent was evaluated by UV-Vis spectrophotometirc method. The dried mixture was ground and sieved through 100 mesh size sieve. The sieved powder and the retained matrix both were evaluated for the content of curcumin at 425 nm.The formulations were then evaluated for drug loading factor.

TABLE 1

Loading factors of curcumin in husk mucilage matrix using various methods in example 1

| SN | Solvent volume 40 ml | | Loading Factor of Curcumin in husk mucilage matrix | | | |
|---|---|---|---|---|---|---|
| | | | Method 1 | Method 2 | Method 3 | Method 4 |
| 1. | Acetone 100% | Ratio of Husk to Curcumin 1:1 | 3.4 | 2.5 | 2.3 | 10.2 |
| 2. | Acetone 75% | | 5.9 | 3.5 | 2.1 | 17.2 |
| 3. | Acetone 50% | | 5.2 | 8.4 | 3.4 | 23 |
| 4. | Acetone 25% | | 10 | 12.94 | 3.9 | 32.9 |
| 5. | Methanol 100% | | 10 | 11 | 6 | 19.2 |
| 6. | Methanol 75% | | 11 | 14 | 4.2 | 28.1 |
| 7. | Methanol 50% | | 12 | 14 | 3.1 | 39 |
| 8. | Methanol 25% | | 14 | 21 | 2.5 | 41.2 |
| 9. | Ethanol 100% | | 20 | 12 | 5 | 25 |
| 10. | Ethanol 75% | | 15 | 18 | 3.1 | 37 |
| 11. | Ethanol 50% | | 10 | 22 | 2.2 | 45 |
| 12. | Ethanol 25% | | 8 | 29 | 1.2 | 68.7 |

As per the results obtained from table 1, 25% ethanol solvent and method 4 [Formulation 12] was selected as the loading factor was observed maximum in this combination.

Swelling factor is the volume in millilitre occupied by 1 g of a drug including any adhering mucilage after it has swollen in an aqueous liquid for 24 h. The swelling factor reflects the mucilage content of the seeds.

TABLE 2

Swelling index of curcumin in husk mucilage matrix for formulation 12
Swelling Index (in ml) of husk matrix in different pH buffer media in 3 hrs

| pH | 1.2 | 3.5 | 4.5 | 6.8 | 7.2 |
|---|---|---|---|---|---|
| Dried husk and Curcumin mixture | 8.8 ± 1.2 | 8.9 ± 1.3 | 9.1 ±0.54 | 10.2 ±0.89 | 7.6 ± 1.23 |

The curcumin release study was also carried out by UV-VIS spectrophotometry 425 nm. The final husk-API mixture (0.1 g) was added to 100ml respective buffer media. The suspension was continuously stirred for 3 hrs.

Procedure:
i. Weighed accurately 0.1 g of sample in a beaker and extracted it with 50 mL of methanol for 30 min.
ii. Filtered the solution in a 100 mL volumetric flask and made up the volume to 100 ml with methanol.
iii. Pipetted out 2.5 mL of the solution in 25 mL volumetric flask and made up the volume with methanol.
iv. From the above diluted solution pipette out 1 mL in 10 mL volumetric flask and made up the volume with methanol.
v. Taken the absorbance of the above solution in UV-spectrophotometer at 425 nm.
vi. Used methanol as a blank solution.

TABLE 3

Release of curcumin in husk mucilage matrix for formulation 12
Release of curcumin (%) from husk matrix in different pH

| pH | 1.2 | 3.5 | 4.5 | 6.8 | 7.2 |
|---|---|---|---|---|---|
| Dried husk- API mixture | 2.1 ± 0.84 | 2.8 ± 1.25 | 2.84 ± 1.32 | 73.4 ± 1.2 | 68.27 ± 0.94 |

Psyllium is very well used in Crohn’s disease as the husk is well swollen in basic ph. Dietary fiber has been proven to be beneficial in maintaining remission in human ulcerative colitis, an effect related with an increased Luminal production of short-chain fatty acids (SCFA). Dietary fiber Supplementation ameliorated colonic damage in HLA-B2712. Intake of psyllium may be effective in alleviating chronic constipation in patients without slow colonic transit or disordered constipation. On the other hand, fiber with lactulose may improve stool consistency in patients with Irritable Bowel

Syndrome (IBS) with constipation. Personality factors influence the magnitude of therapeutic response of the psyllium. The easing of bowel dissatisfaction appears to be a major reason for the therapeutic success of psyllium in IBS.

Additional above-mentioned formulation contains Curcumin with is well stable in gastric pH (because its absorbed in husk) and thus, can exert its effect in colon and ileum area.

The 25% ethanol was the best choice of solvent and the gradual addition under pressure and reduced temperature formulated a stable with more than 65% curcumin loaded psyllium husk powder.

The husk mixture was unaffected by acidic pH, whereas in intestinal pH it released more the 70% of curcumin. Thus, the product can show its effect locally as well as systemically.

We claim:

1. A delayed release composition comprising enteric coated drug loaded in psyllium husk matrix.

2. The composition as claimed in claim 1, wherein the composition drug is curcumin.

3. The composition as claimed in claim 1, wherein the ratio of psyllium husk to drug is 1:1.

4. The composition as claimed in claim 1, wherein the composition comprises a solvent to prepare the matrix.

5. The composition as claimed in claim 4, wherein the solvent is ethanol or methanol or acetone.

6. The composition as claimed in claim 4, wherein the solvent is 25 % ethanol.

7. The composition as claimed in claim 1, wherein the composition provides release of drug in colon and ileum at a pH between 6.8 to 7.2.

8. The composition as claimed in claim 1, wherein the drug loaded in the psyllium husk matrix is more than 65%.

9. The composition as claimed in claim 1, wherein the composition is in the form of granules.

10. A method for preparing a delayed release composition comprising enteric coated drug loaded in psyllium husk matrix, the method comprising
a. Mixing of drug and psyllium husk to form a mixture;
b. Adding solvent to the mixture under pressure to form a matrix;
c. Reducing the temperature of the matrix to allow maximum swelling of mucilage and absorption of the drug into the matrix.

11. The method as claimed in claim 10, wherein the ratio of psyllium husk to drug is 1:1.

12. The method as claimed in claim 10, wherein the solvent is 25% ethanol.

13. The method as claimed in claim 10, wherein the temperature of the matrix is reduced to 10 degree.
